(19) United States
(12) Patent Application Publication
(10) Pub. No.: US 2016/0345937 A1
(43) Pub. Date: Dec. 1, 2016

(54) SYSTEM AND METHOD FOR IMAGING USING ULTRASOUND

(71) Applicant: KONINKLIJKE PHILIPS N.V., Eindhoven (NL)

(72) Inventors: YINHUI DENG, SHANGHAI (CN); WEIPING LIU, SHANGHAI (CN); HUANXIANG LU, SHANGHAI (CN); AMEET KUMAR JAIN, NEW YORK, NY (US); YING WU, SHANGHAI (CN)

(21) Appl. No.: 15/113,875

(22) PCT Filed: Jan. 13, 2015
(86) PCT No.: PCT/EP2015/050439

§ 371 Date: Jul. 25, 2016

(30) Foreign Application Priority Data
Jan. 29, 2014 (CN) PCT/CN2014/071775
May 15, 2014 (EP) 14168404.3

(51) Int. Cl. A61B 8/08; A61B 6/03; A61B 5/055; A61B 8/00; A61B 5/06

(52) U.S. Cl. CPC A61B 8/483; A61B 8/4263; A61B 5/06; A61B 8/5246; A61B 8/5253; A61B 8/5261; A61B 5/055; A61B 6/032

(57) ABSTRACT

The present invention provides a system and a method for imaging a volume of interest of a subject using ultrasound. The system comprises an ultrasound device adapted to acquire an image data set of the volume of interest of the subject and position information of a 3D ultrasound probe of the ultrasound device when the 3D ultrasound probe is placed at a position on the subject, the position information representing a position of the 3D ultrasound probe relative to at least three ultrasound sensors on an interventional device placed within the volume of interest, the at least three ultrasound sensors having predetermined relative positions at a distance from each other and not being aligned in a straight line; and an imaging device adapted to generate an image based on the image data set. According to the system, the position of the ultrasound probe may be derived in a convenient and low-cost manner.

### FIELD OF THE INVENTION

The present invention generally relates to a system and a method for imaging a volume of interest of a subject, e.g., a patient, using ultrasound, especially to positioning an ultrasound probe during the imaging of the volume of interest.

### BACKGROUND OF THE INVENTION

Ultrasound imaging is widely used in clinical applications. Generally it is a free-hand approach. During ultrasound imaging, physicians hold an ultrasound probe and move it on an exterior surface of a subject to scan a plane cutting a volume of interest of the subject.

The positioning of the ultrasound probe would be useful in many clinical applications. Generally, an Electromagnetic (EM) tracking system may be used to determine the position of the ultrasound probe. The EM tracking system comprises an EM sensor attached to the ultrasound probe and an EM field generator which generates an EM field. The position of the EM sensor, i.e., the position of the ultrasound probe, in the EM field may be derived by transmitting an EM signal between the EM field generator and the EM sensor. However, this requires the introduction of an EM tracking system which makes the ultrasound system expensive; and it also requires a registration approach for the EM fields if the system is used at different times.

Another method to determine the position of the ultrasound probe is based on pattern recognition. Although this method has specific requirements with respect to hardware, it is still not reliable.

### SUMMARY OF THE INVENTION

Therefore, it would be desirable to provide a system and a method for imaging a volume of interest of a subject, e.g., a patient, using ultrasound, in which the position of the ultrasound probe may be derived in a convenient and low-cost manner.

According to the present invention, the position of the ultrasound probe may be derived in a coordinate system which is established by using at least three ultrasound sensors having predetermined relative positions at a distance from each other as ultrasound receivers. Since the ultrasound sensors are cheap, it would be a low-cost way of deriving the position of the ultrasound probe.

In addition, according to the present invention, the at least three ultrasound sensors may be attached to an interventional device, such as a needle. When the progress of the insertion of the interventional device into the volume of interest of the subject is monitored in real time by imaging the subject using ultrasound, the at least three ultrasound sensors on the interventional device may be used as reference objects to derive the position of the ultrasound probe during the insertion of the interventional device. There is no need for other reference objects. Since it is the object to be monitored by the ultrasound probe that is used as a reference object for positioning the ultrasound probe, which means that the object to be monitored is the same as the reference object for positioning, it is guaranteed that the reference object for positioning is in the scanning range of the ultrasound probe when the ultrasound probe is positioned such that the object to be monitored or imaged is in the scanning range of the ultrasound probe. Compared with other tracking or locating methods based on other reference objects to be used during the insertion of the interventional device, the method according to the invention is more convenient and/or more reliable. In particular, since the relative positions between the at least three sensors are predetermined, it is not very computationally complex to derive the position information.

In one aspect, the present invention provides a system for imaging a volume of interest of a subject using ultrasound, which comprises an ultrasound device adapted to acquire an image data set of the volume of interest of the subject and position information of a 3D ultrasound probe of the ultrasound device when the 3D ultrasound probe is placed at a position on the subject, the position information representing a position of the 3D ultrasound probe relative to at least three ultrasound sensors on an interventional device being placed within the volume of interest, the at least three ultrasound sensors having predetermined relative positions at a distance from each other and not being aligned in a straight line; and an imaging device adapted to generate an image based on the image data set. The ultrasound device comprises the 3D ultrasound probe adapted to acquire the image data set of the volume of interest, and to sequentially transmit a set of first ultrasound signals for positioning towards the volume of interest, each ultrasound signal of the set of first ultrasound signals for positioning being transmitted along a different scanning line; a receiving unit adapted to receive sensor data from each of the at least three ultrasound sensors; and a positioning unit adapted to derive the position information based on the set of first ultrasound signals for positioning, the sensor data of each of the at least three ultrasound sensors, and the predetermined relative positions of the at least three ultrasound sensors.

Generally, the sensor data received from each ultrasound sensor represents one or more second ultrasound signals received by the corresponding ultrasound sensor. The positioning unit is adapted to select, for each of the at least three ultrasound sensors, a second ultrasound signal having a maximum amplitude among the one or more second ultrasound signals received by the corresponding ultrasound sensor and derive a propagation time of a first ultrasound signal between the 3D ultrasound probe and the corresponding ultrasound sensor based on the selected second ultrasound signal, the set of first ultrasound signals for positioning and the sensor data. Meanwhile, the positioning unit is further adapted to derive position information based on the derived propagation time for each of the at least three ultrasound sensors and the predetermined relative positions of the at least three ultrasound sensors.

In one embodiment, the ultrasound device is adapted to transmit a set of ultrasound signals for imaging towards the volume of interest, and to receive ultrasound echo signals from the volume of interest, and to acquire the image data set of the volume of interest based on the ultrasound echo signals; and the set of ultrasound signals for imaging comprises the set of first ultrasound signals for positioning. In this way, there is no need to transmit/receive additional ultrasound signals for positioning. Rather, at least part of the signal for imaging is simultaneously used for positioning as well. In other words, the monitoring of the insertion of the interventional device and the positioning of the ultrasound probe may be carried out simultaneously. Hence, no extra time is required for positioning.

In one embodiment, the imaging device is further adapted to obtain positions of the at least three ultrasound sensors in a coordinate system of a different imaging modality and generate an image by fusing the image and an image of the different imaging modality based on the derived position information of the 3D ultrasound probe and the positions of the at least three ultrasound sensors in the coordinate system of the different imaging modality. The different imaging modality is any one of CT, X-Ray and MRI.

In another embodiment, the ultrasound device is further adapted to acquire a first image data set of the volume of interest and first position information of the 3D ultrasound probe when the 3D ultrasound probe is placed at a first position on the subject, and to acquire a second image data set of the volume of interest and second position information of the 3D ultrasound probe when the 3D ultrasound probe is placed at a second position on the subject. Meanwhile, the imaging device is further adapted to generate the image by combining the first image data set and the second image data set based on the first position information and the second position information.

In a further embodiment, the ultrasound device is further adapted to acquire a first image data set of the volume of interest and first position information of the 3D ultrasound probe when the 3D ultrasound probe is placed at a first position on the subject and the at least three sensors are placed at first sensor positions, and to acquire a second image data set of the volume of interest and second position information of the 3D ultrasound probe when the 3D ultrasound probe is placed at a second position and the at least three sensors are placed at second sensor positions. Meanwhile, the imaging device is further adapted to generate the image by combining the first image data set and the second image data set based on the first position information, the second position information and a relative position between the first sensor positions and the second sensor positions.

As described in the above, the derived position information about the ultrasound probe can be used to combine an ultrasound image with an image of a different modality, such as CT, X-Ray and MRI, or combine two or more ultrasound images.

In another aspect, the present invention provides a method of imaging a volume of interest of a subject using ultrasound, wherein a 3D ultrasound probe is adapted to acquire an image data set of the volume of interest, and to sequentially transmit a set of first ultrasound signals for positioning towards the volume of interest, each ultrasound signal of the set of first ultrasound signals for positioning being transmitted along a different scan line, the method comprising the following steps: receiving sensor data from each of at least three ultrasound sensors on an interventional device placed within the volume of interest, the at least three ultrasound sensors having predetermined relative positions at a distance from each other and not being aligned in a straight line, deriving position information of the 3D ultrasound probe based on the set of first ultrasound signals for positioning, the sensor data of each of the at least three ultrasound sensors, and the predetermined relative positions of the at least three ultrasound sensors, the position information representing a position of the 3D ultrasound probe relative to at least three ultrasound sensors; and generating an image based on the image data set.

In a further aspect, the present invention provides a computer program product comprising computer program instructions for performing the method according to the invention when it is performed by a processor.

Various aspects and features of the disclosure are described in further detail below. And other objects and advantages of the present invention will become more apparent and will be easily understood with reference to the description made in combination with the accompanying drawings.

### DESCRIPTION OF THE DRAWINGS

The present invention will be described and explained hereinafter in more detail in combination with embodiments and with reference to the drawings, wherein:

FIG. 1 is a schematic block diagram of a system 1 for imaging a volume of interest of a subject using ultrasound according to an embodiment of the present invention;

FIGS. 2a and 2b are schematic representations of sensor signals S and S′ and corresponding ultrasound signals for positioning according to the present invention;

FIG. 3 is a flowchart of a method for imaging a volume of interest of a subject using ultrasound according to an embodiment of the present invention.

The same reference signs in the figures indicate similar or corresponding features and/or functionalities.

### DETAILED DESCRIPTION

The present invention will be described with respect to particular embodiments and with reference to certain drawings, but the invention is not limited thereto but only by the claims. The drawings described are only schematic and are non-limiting. In the drawings, the size of some of the elements may be exaggerated and not drawn to scale for illustrative purposes.

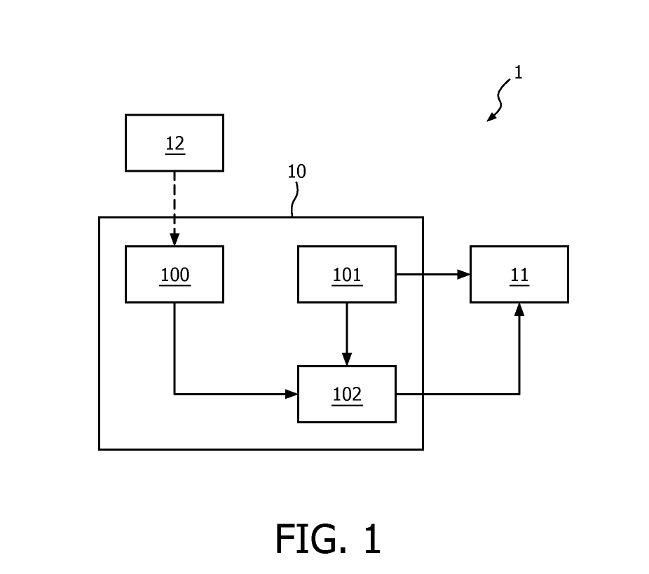

FIG. 1 is a schematic block diagram of a system 1 for imaging a volume of interest of a subject using ultrasound, e.g., a patient, according to an embodiment of the present invention. The ultrasound imaging system 1 comprises an ultrasound device 10 for acquiring an image data set of the volume of interest of the subject and position information of an ultrasound probe 101, in particular a 3D ultrasound probe, of the ultrasound device 10 when the ultrasound probe 101 is placed at a position on the subject and an imaging device 11 for generating an image of the volume of interest of the subject based on the image data set of the volume of interest of the subject.

As shown in FIG. 1, the ultrasound device 10 comprises a 3D ultrasound probe 101 which may be placed on the subject at a position and which transmits a set of ultrasound signals towards the volume of interest of the subject. The set of ultrasound signals may be transmitted sequentially along different scan lines. The set of ultrasound signals may be a set of ultrasound signals for positioning the 3D ultrasound probe 101 or a set of ultrasound signals for imaging the volume of interest of the subject. At least part of the set of ultrasound signals for imaging may also be used as the ultrasound signals for positioning the 3D ultrasound probe 101. In this way, it is possible that one set of ultrasound signals is used for both imaging and positioning. This would reduce the time necessary for imaging the volume of interest and positioning the 3D ultrasound probe. The ultrasound device 10, especially the 3D ultrasound probe 101, receives ultrasound echo signals from the volume of interest of the subject and acquires the image data set of the volume of interest based on the received ultrasound echo signals.

The ultrasound device 10 further comprises a receiving unit 100, e.g., an interface unit, which receives sensor data from each of the at least three ultrasound sensors 12 and transmits the data to a positioning unit 102. Alternatively, the receiving unit 100 and the positioning unit 102 can be separate from the ultrasound device 10 but part of the system 1 and they may be in communication with the ultrasound device 10.

The at least three ultrasound sensors 12 may be attached to an interventional device within the volume of interest of the subject and occupy predetermined relative positions at a distance from each other. The interventional device may be a rigid device such as a needle in which the relative positions of the at least three ultrasound sensors 12 may be kept unchanged during the progress of the insertion of the interventional device into the subject. It may also be possible that the interventional device is a flexible device, such as a catheter, on which the at least three ultrasound sensors 12 are attached at predetermined relative positions at a distance from each other during the progress of the insertion of the interventional device into the subject, for example by means of a rigid fixture.

In one embodiment, the distance between any two of the at least three ultrasound sensors 12 is to be predetermined.

It may be noted that the at least three ultrasound sensors 12 are not aligned in a straight line. As known to the skilled person, the ultrasound sensor is very small, and so it is possible to arrange multiple ultrasound sensors so as to be not aligned in a straight line on an interventional device, including a needle. In some cases, the at least three ultrasound sensors 12 may be receivers of the ultrasound signals only. Since the receivers of the ultrasound signals may be much cheaper than the ultrasound transducer used for both transmitting and receiving, a cost-efficient manner of positioning 3D ultrasound probe 101 would be provided.

The sensor data received by the receiving unit 100 represents one or more second ultrasound signals received by each ultrasound sensor 12. The one or more second ultrasound signals are received in response to the transmitting of one or more first ultrasound signals of the set of first ultrasound signals for positioning from the ultrasound probe 101.

Please note that the first ultrasound signal refers to an ultrasound signal transmitted by the ultrasound probe 101 and the second ultrasound signal refers to an actually received ultrasound signal by the ultrasound sensor 12 in response to the transmitting of a corresponding first ultrasound signal.

Although the ultrasound signals actually received by the ultrasound sensor 12 and the ultrasound signals transmitted by the ultrasound probe 101 for positioning may be correlated with each other, they may be slightly different from each other. In particular, the ultrasound signals transmitted by the ultrasound probe 101 along scan lines adjacent to an ultrasound sensor 12 may be received by the ultrasound sensor 12 also. In an embodiment, the amplitudes of the ultrasound signals actually received by the ultrasound sensor 12 for the adjacent scan lines would be smaller than those of the corresponding ultrasound signals transmitted by the ultrasound probe 101. In the context of the description, the first ultrasound signal(s) and the second ultrasound signal(s) are used for distinguishing between the ultrasound signals actually received by the ultrasound sensor 12 and the ultrasound signals transmitted by the ultrasound probe 101 for positioning.

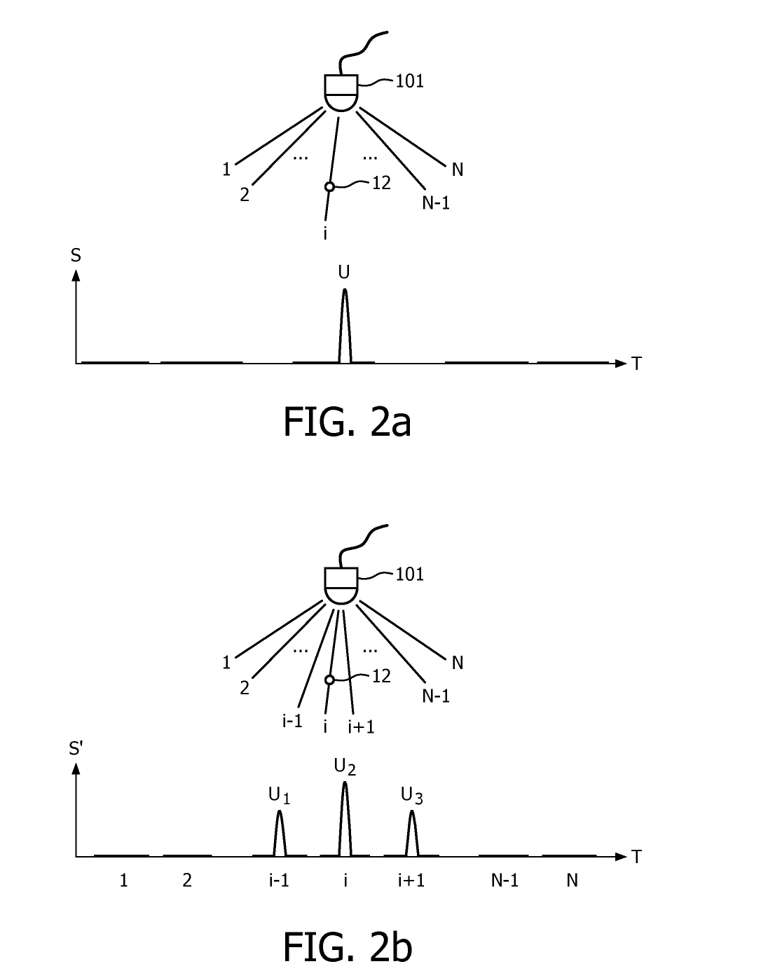

FIG. 2a shows a sensor data S and a set of corresponding first ultrasound signals for positioning according to the present invention. As shown in FIG. 2a, an ultrasound probe 101 transmits a set of first ultrasound signals for positioning along different scan lines 1, 2, . . . , i, . . . , N−1, N towards the volume of interest of the subject, and an ultrasound sensor 12, which is located along a scan line i, generates a sensor data S in response to receiving, by the ultrasound sensor 12, one or more corresponding first ultrasound signals of the set of first ultrasound signals for positioning.

According to the sensor data S shown in FIG. 2a, the y axis indicates the amplitude of the second ultrasound signal(s) received by a corresponding ultrasound sensor 12 and the x axis indicates the time at which the ultrasound sensor 12 receives second ultrasound signals in response to the transmitting of the first ultrasound signals along the scan lines 1, 2, . . . , i, . . . , N−1, N towards the volume of interest of the subject.

It may be preferred that the ultrasound sensor 12 receives only the ultrasound signal transmitted towards it by the ultrasound probe 101. That is, the ultrasound sensor 12 does not receive ultrasound signals transmitted along scan lines adjacent thereto. Assuming that an ultrasound sensor 12 is located along a scan line i, when a first ultrasound signal is transmitted by the ultrasound probe 101 along the scan line i, the ultrasound sensor 12 may receive a second ultrasound signal. In contrast, no second ultrasound signals may be received by the ultrasound sensor 12 when the ultrasound probe 101 transmits first ultrasound signals along scan lines other than the scan line i. According to the sensor data S shown in FIG. 2a, a second ultrasound signal U is shown corresponding to the scan line i, while second ultrasound signals corresponding to other scan lines are not shown.

Alternatively, the first ultrasound signals transmitted along scan lines adjacent to an ultrasound sensor 12 may be received by the ultrasound sensor 12 as well. This is shown in FIG. 2b, in which a sensor data S′ may be obtained by the ultrasound sensor 12 located along the scan line i. In this embodiment, the ultrasound sensor 12 may also receive first ultrasound signals transmitted by the ultrasound probe 101 along scan lines i−1 and i+1. However, since the first ultrasound signals transmitted along the scan lines i−1 and i+1 are not directed to the ultrasound sensor 12 directly, the second ultrasound signals received by the ultrasound sensor 12 in response to the transmitting of first ultrasound signals along the scan lines i−1 and i+1 may have a smaller amplitude than the second ultrasound signal received by the ultrasound sensor 12 in response to the transmitting of a first ultrasound signal along the scan line i. This is shown in FIG. 2b, in which the amplitude of a received second ultrasound signal U₂ corresponding to the scan line i at which the ultrasound sensor is located is larger than that of second ultrasound signals U₁ and U₃ corresponding to the adjacent scan lines i−1 and i+1.

FIGS. 2a and 2b show the case where one sensor data S, S′ is generated by one ultrasound sensor 12. In fact, a sensor data may be obtained for each of the at least three ultrasound sensors 12 individually and transmitted to the receiving unit 100.

Both of the sensor data received by the receiving unit 100 and the set of first ultrasound signals transmitted by the ultrasound probe 101 are transmitted to a positioning unit 102. The positioning unit 102 derives position information representing a position of the ultrasound probe 101 relative to the at least three ultrasound sensors 12 based on the set of first ultrasound signals, the sensor data received from each of the at least three ultrasound sensors 12, and the predetermined relative positions of the at least three ultrasound sensors 12.

In particular, the positioning unit 102 selects a second ultrasound signal having a maximum amplitude among the one or more second ultrasound signals received by each ultrasound sensor 12 based on the sensor data for the corresponding ultrasound sensor 12, derives a propagation time of a first ultrasound signal from the ultrasound probe 101 to the corresponding ultrasound sensor 12 based on the selected second ultrasound signal, and the set of first ultrasound signals and the sensor data, and derives the position information of the 3D ultrasound probe 101 based on the derived propagation time for each of the at least three ultrasound sensors and the predetermined relative positions of the at least three ultrasound sensors 12.

As shown in FIG. 2b, for a sensor data S′, a second ultrasound signal U₂ is selected as having a maximum amplitude among the one or more second ultrasound signals U₁, U₂ and U₃, based on the selected second ultrasound signal U₂ and the set of first ultrasound signals, a first ultrasound signal of the set of first ultrasound signals transmitted along the scan line i is selected as corresponding to the selected second ultrasound signal, and the first ultrasound signal is selected for determining the propagation time thereof.

In the case where the corresponding ultrasound sensor receives directly one second ultrasound signal U corresponding to a first ultrasound signal transmitted along a scan line i, as shown in FIG. 2a, the first ultrasound signal transmitted along the scan line i is selected for deriving the propagation time directly.

The propagation time of the first ultrasound signal from the ultrasound probe 101 to the corresponding ultrasound sensor 12 can be derived based on the selected second ultrasound signal, the set of first ultrasound signals and the sensor data by means of various approaches. For example, the ultrasound device 10 may additionally include a recording unit (not shown) which records the timing at which the ultrasound probe 101 sequentially transmits the set of ultrasound signals towards the volume of interest of the subject and the sensor data includes timing information representing the timing at which the corresponding ultrasound sensor receives the second ultrasound signals. The approach to derive the propagation time of the first ultrasound signal is known to those skilled in the art, the description given above is only for illustration, but not for limitation. Those skilled in the art may also use other methods for deriving the propagation time.

After the propagation time is derived for each of the at least three ultrasound sensors, the positioning unit 102 may determine distances between the ultrasound probe 101 and each of the at least three ultrasound sensors 12 based on the derived propagation time for corresponding ultrasound sensors 12 and propagation velocity of the ultrasound signal in the subject.

Based on the determined distances between the ultrasound probe 101 and each of the at least three ultrasound sensors 12 and the predetermined relative positions of the at least three ultrasound sensors 12, the position information of the ultrasound probe 101 may be derived by solving an equation system. For persons skilled in mathematics, it may be easy to establish an equation system for solving a position based on the known position relationships between the position and at least three positions, the at least three positions having predetermined relative relationships. During solving the equation system, the scan line along which the selected ultrasound signal is transmitted may be used also.

Referring back to FIG. 1, the ultrasound imaging system 1 may also include an imaging device 11 which may receive the image data set from the ultrasound device 10, in particular the 3D ultrasound probe 101, and optionally the position information of the 3D ultrasound probe 101 from the positioning unit 102. In some cases, the imaging device 11 may generate an image based on both an image data set and the position information. In particular, the imaging device 11 may generate an image by fusing an ultrasound image generated based on the image data set received from the 3D ultrasound probe 101 with an image of a different image modality or by combining a plurality of ultrasound image data sets each received from the 3D ultrasound probe 101 when the 3D ultrasound probe 101 is placed at a different position on the subject based on corresponding position information of the 3D ultrasound probe 101.

In some cases, it is desirable to fuse an ultrasound image and an image of a different imaging modality, such as any one of CT, X-Ray and MRI. The imaging device may obtain positions of the at least three ultrasound sensors in a coordinate system of a different imaging modality and generate an image by fusing the image and an image of the different imaging modality based on the derived position information of the 3D ultrasound probe (101) and the obtained positions of the at least three ultrasound sensors (12). According to this embodiment, the skilled person would understand that the position of the ultrasound probe in the coordinate system of the different imaging modality can be known from the relative position between the ultrasound probe and the at least three ultrasound sensors and the position of the at least three ultrasound sensors in the coordinate system of the other imaging modality, and the fusing of the ultrasound image and the image of the different imaging modality can be simplified and/or improved in accuracy by knowing the position of the ultrasound probe in the coordinate system of the different imaging modality. For example, the positions of the at least three ultrasound sensors in a coordinate system can be the positions of the at least three ultrasound sensors relative to the source and detector of the different imaging modality.

In another embodiment, as the 3D ultrasound probe 101 moves between a plurality of positions on the subject when the interventional device is fixed at a point, i.e., the positions of the at least three ultrasound sensors is unchanged, a plurality of image data sets may be obtained for the plurality of positions of the 3D ultrasound probe 101. For example, as the ultrasound probe 101 moves on a subject from a first position to a second position, the ultrasound device 10 may acquire a first ultrasound image data set of a volume of interest of the subject and first position information of the ultrasound probe 101 when the ultrasound probe 101 is placed at the first position, and a second ultrasound image data set of the volume of interest of the subject and second position information of the ultrasound probe 101 when the ultrasound probe 101 is placed at the second position, as described above. The first position information represents the position of the ultrasound probe 101 relative to the at least three ultrasound sensors when the ultrasound probe 101 is placed at the first position, and the second position information represents the position of the ultrasound probe 101 relative to the at least three ultrasound sensors when the ultrasound probe 101 is placed at the second position.

In this case, the imaging device 11 receives the first ultrasound image data set, the second ultrasound image data set, the first position information and the second position information and generates an image by combining the first ultrasound image data set and the second ultrasound image data set based on the first position information and the second position information. Although the above description only refers to the case where the ultrasound probe 101 is placed at two positions sequentially, it may be obvious that the ultrasound probe 101 may also be placed at more than two positions sequentially.

This would be very beneficial when a large object is imaged using an ultrasound probe with a limited view scope. The ultrasound probe 101 may move between a plurality of positions and obtain an image data set of a part of the object for each of the plurality of positions. Based on the position information of the ultrasound probe 101 that is determined for each of the plurality of positions by using the approach as described above, an image for the large object may be generated via the imaging device 11 by combining image data sets generated when the ultrasound probe is placed at different positions.

In a further embodiment, during the progress of the insertion of the interventional device into the volume of interest of the subject, the positions of the at least three ultrasound sensors may be varied also since the at least three ultrasound sensors are attached to the interventional device. For example, the at least three ultrasound sensors are moved from first sensor positions to second sensor positions as the interventional device moves. In order to monitor the interventional device in the volume of interest of the subject, the ultrasound probe 101 may be moved accordingly from a first position to a second position on the subject to image the volume of interest and the interventional device.

The ultrasound device 10 acquires a first image data set of the volume of interest and first position information of the ultrasound probe 101 relative to the at least three ultrasound sensors when the 3D ultrasound probe 101 is placed at the first position on the subject and the at least three sensors 12 are placed at the first sensor positions, and it acquires a second image data set of the volume of interest and second position information of the ultrasound probe 101 relative to the at least three ultrasound sensors when the 3D ultrasound probe 101 is placed at the second position and the at least three sensors 12 are placed at second sensor positions.

In this case, the imaging device 11 may combine the first image data set and the second image data set based on the first position information, the second position information and the relative position between the first sensor positions and the second sensor positions of the at least three ultrasound sensors 12, and generate an image based on the combined first image data set and second image data set. Those skilled in the art would understand that it is not necessary to obtain the first sensor positions and the second sensor positions of the at least three ultrasound sensors 12, because the technical solution of this embodiment may be achieved by knowing the relative position between the first sensor positions and the second sensor positions. In an embodiment, the relative position between the first sensor positions and the second sensor positions of the at least three ultrasound sensors 12 can be provided by a tracking device/system for tracking the position of the interventional device to which the at least three ultrasound sensors are attached.

Although the system of ultrasound imaging of the invention is described with respect to an ultrasound device 10 comprising a receiving unit 100, an ultrasound probe 101 and a positioning unit 102, and an imaging device 11, as shown in FIG. 1, it may be determined that the system of the invention is not limited to the configurations described above. One or more units or components of the system may be omitted or integrated into one component to perform the same function. For example, the receiving unit 100 may be integrated with the positioning unit 102 to combine its function with that of(?) the positioning unit 102. Alternatively, the units or components of the system of the invention may also be further divided into different units or components, for example, the positioning unit 102 may be divided into several separate units to perform corresponding functions. Furthermore, it may be determined that the receiving unit 100, the positioning unit 102, and the imaging device 11 of the system of the invention may be achieved by means of any one of software, hardware, firmware or a combination thereof. In particular, they may be achieved not only by computer programs for performing corresponding functions but also by various entity devices, such as application-specific integrated circuits (ASIC), digital signal processors (DLP), programmable logical devices (PLD), field-programmable gate arrays (FPGA), and CPUs. Although the receiving unit 100 and the positioning unit 102 are shown as part of the ultrasound device 10 and the imaging device 11 is shown as a separate device from the ultrasound device 10 in FIG. 1, this is only for the purpose of illustration of the invention, but not for limitation. It may be understood that the receiving unit 100, the positioning unit 102, and the imaging device 11 may be randomly combined or divided as long as the corresponding functions can be achieved.

Although the operation of the imaging device 11 has been described with respect to different cases as described above, it may be contemplated that the imaging device 11 may also generate an ultrasound image based on an image data set acquired when an ultrasound probe is placed at one position only, i.e., generate the ultrasound image when the ultrasound probe is placed at one position only. In this case, the imaging device 11 does not need to receive the position information of the ultrasound probe 101 from the positioning unit 102. The positioning unit 102 may output the position information of the ultrasound probe 101 to a display. This would be beneficial for applying an ultrasound imaging guidance approach according to a plan, which is required to have the position information of the ultrasound probe 101 and then the physicians may follow the plan.

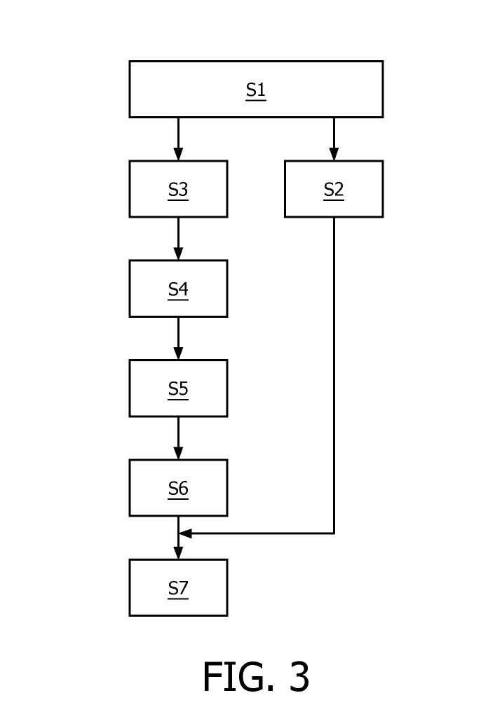

FIG. 3 shows a flowchart of a method for imaging a volume of interest of a subject using ultrasound according to an embodiment of the present invention. In step S1, an ultrasound probe 101, e.g., a 3D ultrasound probe, is placed at a position on a subject and a set of ultrasound signals is transmitted by the 3D ultrasound probe 101 towards a volume of interest of the subject along different scan lines. The set of ultrasound signals may be a set of first ultrasound signals for positioning or a set of ultrasound signals for imaging which comprises the set of first ultrasound signals for positioning.

In step S2, ultrasound echo signals are received from the volume of interest by the 3D ultrasound probe 101 and an image data set of the volume of interest is acquired based on the received ultrasound echo signals.

In step S3, in response to the transmitted first ultrasound signals from the ultrasound probe 101, each of at least three ultrasound sensors 12 generates a corresponding sensor data S, S′. The at least three ultrasound sensors 12 are attached to an interventional device placed within the volume of interest, have predetermined relative positions at a distance from each other and are not aligned in a straight line. The sensor data S, S′ represents one or more second ultrasound signals U, U₁, U₂, U₃ received by the corresponding ultrasound sensor 12. The sensor data S, S′ of each of at least three ultrasound sensors 12 is received by the receiving unit 101.

In steps S4-S6, position information of the ultrasound probe 101 may be derived based on the set of first ultrasound signals for positioning, the sensor data S, S′ of each of the at least three ultrasound sensors 12, and the predetermined relative positions of the at least three ultrasound sensors 12, the position information representing a position of the ultrasound probe 101 relative to the at least three ultrasound sensors 12.

In particular, in step S4, for each of the at least three ultrasound sensors 12, a second ultrasound signal having a maximum amplitude among the one or more second ultrasound signals received by the corresponding ultrasound sensor, is selected based on the sensor data S, S′. As shown in FIG. 2b, a second ultrasound signal U₂ represented by sensor data S′ is selected among the second ultrasound signals U₁, U₂ and U₃, since it has a maximum amplitude.

In step S5, for each of the at least three ultrasound sensors, a propagation time of a first ultrasound signal between the 3D ultrasound probe 101 and the corresponding ultrasound sensor 12 may be derived based on the selected second ultrasound signal, the set of first ultrasound signals for positioning and the sensor data S, S′, as described above.

In step S6, the position information of the ultrasound probe 101 may be derived based on the derived propagation time for each of the at least three ultrasound sensors and the predetermined relative positions of the at least three ultrasound sensors 12.

In particular, distances between each of the at least three ultrasound sensors 12 and the ultrasound probe 101 may be derived based on the derived propagation time for the corresponding ultrasound sensors, and the position information of the ultrasound probe 101 may be derived by establishing and solving an equation system based on the distances between each of the at least three ultrasound sensors 12 and the ultrasound probe 101 and the predetermined relative positions of the at least three ultrasound sensors 12. As described above, the scan line along which the selected ultrasound signal is transmitted may be used also for solving the equation system.

In step S7, the position information of the ultrasound probe 101 and the image data set are received by the imaging device 11 and an image is generated based thereon.

According to different cases where the system and method of the invention is used, an image may be generated by the imaging device 11 by fusing an ultrasound image with an image of a different imaging modality or by combining a plurality of ultrasound image data sets based on the position information of the ultrasound probe 101.

In one case, an image is generated by the imaging device 11 by fusing an ultrasound image generated from the image data set acquired by the ultrasound probe 101 and an image of a different imaging modality, such as any one of CT, X-Ray and MRI, based on the derived position information of the ultrasound probe 101 and the positions of the at least three ultrasound sensors 12 in a coordinate system of the different imaging modality. The positions of the at least three ultrasound sensors (12) may be obtained by the imaging device 11. For example, the positions of the at least three ultrasound sensors (12) in the different imaging modality can be provided by a device/system for providing the image of the different imaging modality.

In another case, the ultrasound probe 101 moves from a first position to a second position on the subject while the positions of the at least three ultrasound sensors remain unchanged. In this case, in step S2, the ultrasound probe 101 acquires a first image data set of the volume of interest when the ultrasound probe 101 is placed at the first position on the subject and a second image data set of the volume of interest when the ultrasound probe 101 is placed at the second position on the subject. In steps S3-S6, first position information of the ultrasound probe 101 when the 3D ultrasound probe 101 is placed at the first position on the subject and second position information of the 3D ultrasound probe 101 when the 3D ultrasound probe 101 is placed at the second position on the subject are derived. In step S7, the imaging device generates an image by combining the first image data set and the second image data set based on the first position information and the second position information.

In a further case, the at least three ultrasound sensors move from first sensor positions to second sensor positions as the interventional device on which the at least three ultrasound sensors are attached moves in the volume of interest, and the ultrasound probe 101 moves accordingly from a first position to a second position on the subject in order to monitor the movement of the interventional device in the volume of interest.

In this case, in step S2, a first image data set of the volume of interest is acquired by the ultrasound probe 101 when the ultrasound probe 101 is placed at the first position on the subject and the at least three sensors are placed at the first sensor positions and a second image data set of the volume of interest is acquired by the ultrasound probe 101 when the ultrasound probe 101 is placed at the second position on the subject and the at least three sensors are placed at the second sensor positions.

In steps S3-S6, first position information of the ultrasound probe is derived when the ultrasound probe 101 is placed at the first position on the subject and the at least three sensors are placed at the first sensor positions and second position information of the ultrasound probe 101 is derived when the ultrasound probe 101 is placed at the second position on the subject and the at least three sensors are placed at the second sensor positions.

In step S7, an image is generated by the imaging device 11 by combining the first image data set and the second image data set based on the first position information, the second position information and a relative position between the first sensor positions and the second sensor positions of the at least three ultrasound sensors.

Although the method of the invention is described with respect to the steps S1-S7 shown in FIG. 3, it may be understood that some of the steps may be integrated or sub-divided as long as the corresponding functions can be achieved.

It may also be contemplated that an ultrasound image is generated only based on an image data set acquired when an ultrasound probe is placed at a position in step S7. In this case, in step S7, only one image data set is received and there is no need to receive the position information of the ultrasound probe 101 from step S6. The ultrasound image generated in step S7 and the position information of the ultrasound probe 101 generated in step S6 may be sent to a display (not shown) for display thereof.

Please note that the device according to the present invention should not be limited to that mentioned above. It will be apparent to those skilled in the art that the various aspects of the invention claimed may be practiced in other examples that depart from these specific details.

Furthermore, the mere fact that certain measures are recited in mutually different dependent claims does not indicate that a combination of these measures cannot be used to advantage.

It should be noted that the above-mentioned embodiments illustrate rather than limit the invention and that those skilled in the art would be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. In the product claims enumerating several units, several of these units can be embodied by one and the same item of software and/or hardware. The usage of the words first, second and third, et cetera, does not indicate any ordering. These words are to be interpreted as names.

1. A system for imaging a volume of interest of a subject using ultrasound, comprising:
an ultrasound device adapted to acquire an image data set of the volume of interest of the subject and position information of a 3D ultrasound probe of the ultrasound device when the 3D ultrasound probe is placed at a position on the subject, the position information representing a position of the 3D ultrasound probe relative to at least three ultrasound sensors on an interventional device placed within the volume of interest, the at least three ultrasound sensors having predetermined relative positions at a distance from each other and not being aligned in a straight line; and
an imaging device adapted to generate an image based on the image data set; wherein the ultrasound device comprises:
the 3D ultrasound probe adapted to acquire the image data set of the volume of interest, and to sequentially transmit a set of first ultrasound signals for positioning towards the volume of interest, each ultrasound signal of the set of first ultrasound signals for positioning being transmitted along a different scan line (1, 2, . . . , i, . . . , N);
a receiving unit adapted to receive sensor data from each of the at least three ultrasound sensors;
a positioning unit adapted to derive the position information based on the set of first ultrasound signals for positioning, the sensor data of each of the at least three ultrasound sensors, and the predetermined relative positions of the at least three ultrasound sensors.

2. The system of claim 1, wherein
the sensor data received from each ultrasound sensor represents one or more second ultrasound signals received by the corresponding ultrasound sensor;
the positioning unit is adapted to select, for each of the at least three ultrasound sensors, a second ultrasound signal having a maximum amplitude among the one or more second ultrasound signals received by the corresponding ultrasound sensor and derive a propagation time of a first ultrasound signal between the 3D ultrasound probe and the corresponding ultrasound sensor based on the selected second ultrasound signal, the set of first ultrasound signals for positioning and the sensor data; and
the positioning unit is further adapted to derive the position information based on the derived propagation time for each of the at least three ultrasound sensors and the predetermined relative positions of the at least three ultrasound sensors.

3. The system of claim 1, wherein
the ultrasound device is adapted to transmit a set of ultrasound signals for imaging towards the volume of interest, and to receive ultrasound echo signals from the volume of interest, and to acquire the image data set of the volume of interest based on the ultrasound echo signals; and
the set of ultrasound signals for imaging comprises the set of first ultrasound signals for positioning.

4. The system of claim 1, wherein the imaging device is further adapted to obtain positions of the at least three ultrasound sensors in a coordinate system of a different imaging modality and generate an image by fusing the image and an image of the different imaging modality based on the derived position information of the 3D ultrasound probe and the obtained positions of the at least three ultrasound sensors (12).

5. The system of claim 4, wherein the other imaging modality is any one of CT, X-Ray and MRI.

6. The system of claim 1, wherein
the ultrasound device is further adapted to acquire a first image data set of the volume of interest and first position information of the 3D ultrasound probe when the 3D ultrasound probe is placed at a first position on the subject, and to acquire a second image data set of the volume of interest and second position information of the 3D ultrasound probe when the 3D ultrasound probe is placed at a second position on the subject; and
the imaging device is further adapted to generate the image by combining the first image data set and the second image data set based on the first position information and the second position information.

7. The system of claim 1, wherein
the ultrasound device is further adapted to acquire a first image data set of the volume of interest and first position information of the 3D ultrasound probe when the 3D ultrasound probe is placed at a first position on the subject and the at least three sensors are placed at first sensor positions, and to acquire a second image data set of the volume of interest and second position information of the 3D ultrasound probe when the 3D ultrasound probe is placed at a second position and the at least three sensors are placed at second sensor positions; and
the imaging device is further adapted to generate the image by combining the first image data set and the second image data set based on the first position information, the second position information and a relative position between the first sensor positions and the second sensor positions.

8. A method of imaging a volume of interest of a subject using ultrasound, wherein a 3D ultrasound probe is adapted to place at a position on a subject and acquire an image data set of the volume of interest, and to sequentially transmit a set of first ultrasound signals for positioning towards the volume of interest, each ultrasound signal of the set of first ultrasound signals for positioning being transmitted along a different scan line (1, 2 . . . , i, . . . , N), the method comprising the following steps:
receiving sensor data from each of at least three ultrasound sensors on an interventional device placed within the volume of interest, the at least three ultrasound sensors having predetermined relative positions at a distance(?) from each other and not being aligned in a straight line;
deriving position information of the 3D ultrasound probe based on the set of first ultrasound signals for positioning, the sensor data of each of the at least three ultrasound sensors, and the predetermined relative positions of the at least three ultrasound sensors, the position information representing a position of the 3D ultrasound probe relative to at least three ultrasound sensors; and
generating an image based on the image data set.

9. The method of claim 8, wherein the received sensor data represents one or more second ultrasound signals received by the corresponding ultrasound sensor and the step of deriving the position information of the 3D ultrasound probe, further comprising:
for each of the at least three ultrasound sensors, selecting a second ultrasound signal (U₂) having a maximum amplitude among the one or more second ultrasound signals received by the corresponding ultrasound sensor, based on the sensor data;
for each of the at least three ultrasound sensors, deriving a propagation time of a first ultrasound signal between the 3D ultrasound probe and the corresponding ultrasound sensor, based on the selected second ultrasound signal, the set of first ultrasound signals for positioning and the sensor data; and
deriving the position information based on the derived propagation time for each of the at least three ultrasound sensors and the predetermined relative positions of the at least three ultrasound sensors.

10. The method of claim 8, further comprising:
transmitting a set of ultrasound signals for imaging towards the volume of interest by the 3D ultrasound probe, the set of ultrasound signals for imaging comprising the set of first ultrasound signals for positioning;
receiving ultrasound echo signals from the volume of interest, and;
acquiring the image data set of the volume of interest based on the ultrasound echo signals.

11. The method of claim 8, further comprising:
obtaining positions of the at least three ultrasound sensors in a coordinate system of a different imaging modality; and
generating an image by fusing the image and an image of the different imaging modality based on the derived position information of the 3D ultrasound probe and the obtained positions of the at least three ultrasound sensors.

12. The method of claim 11, wherein the different imaging modality is any one of CT, X-Ray and MRI.

13. The method of claim 8, further comprising:
acquiring a first image data set of the volume of interest and first position information of the 3D ultrasound probe when the 3D ultrasound probe is placed at a first position on the subject;
acquiring a second image data set of the volume of interest and second position information of the 3D ultrasound probe when the 3D ultrasound probe is placed at a second position on the subject; and
generating the image by combining the first image data set and the second image data set based on the first position information and the second position information.

14. The method of claim 8, further comprising:
acquiring a first image data set of the volume of interest and first position information of the 3D ultrasound probe when the 3D ultrasound probe is placed at a first position on the subject and the at least three sensors are placed at first sensor positions;
acquiring a second image data set of the volume of interest and second position information of the 3D ultrasound probe when the 3D ultrasound probe is placed at a second position on the subject and the at least three sensors are placed at second sensor positions; and
generating the image by combining the first image data set and the second image data set based on the first position information, the second position information and a relative position between the first sensor positions and the second sensor positions.

15. A computer program product comprising computer program instructions for performing the method of claim 8 when it is performed by a processor.
